(19) United States
(12) Patent Application Publication
(10) Pub. No.: US 2018/0230498 A1
(43) Pub. Date: Aug. 16, 2018

(54) High Protein Animal Feed Byproduct From Cereal - Pulse Feedstock Blend Ethanol Production

(71) Applicant: Prairie Green Renewable Energy Inc., Calgary (CA)

(72) Inventors: Ernest Holinaty, Porcupine Plains (CA); Ernest Nycz, Calgary (CA); Mark Vandaele, Medira (CA)

(21) Appl. No.: 15/894,862

(22) Filed: Feb. 12, 2018

Related U.S. Application Data
(60) Provisional application No. 62/457,974, filed on Feb. 12, 2017.

(51) Int. Cl. C12P 7/06; A23K 10/38

(52) U.S. Cl. CPC C12P 7/06; A23K 10/38

(57) ABSTRACT

In one aspect there is provided a method for producing ethanol and distiller's dried grains and solubles (DDGS). This method comprises the steps of: providing a plurality of feedstocks; milling said feedstocks; mixing the milled feedstocks into a feedstock blend; producing ethanol from the feedstock blend using a dry milling process; and collecting the DDGS by-product, after the ethanol is produced. In another aspect, the method further comprises providing barley as one of the feedstocks and peas as a second feedstock, wherein the barley feedstock and pea feedstock is mixed in a ratio of between 2:1 and 3.5:1 to make the feedstock blend. In yet another embodiment, antifoaming enzymes are added during the dry milling process to reduce foaming during the fermentation stage of the dry mill process.

### CROSS REFERENCE TO RELATED APPLICATION

This application is a non-provisional application which claims priority to, and benefit of, U.S. provisional patent application Ser. No. 62/457,974 filed Feb. 12, 2017 and entitled, “High Protein Animal Feed Byproduct from Cereal—Pulse Feedstock Blend Ethanol Production”, the entirety of which is incorporated herein by reference.

### FIELD

This invention relates generally to producing ethanol from a feedstock. More particularly, the invention relates to a method of producing ethanol and a high protein distiller's dried grains and solubles (DDGS) by-product.

### BACKGROUND

The background information discussed below is presented to better illustrate the novelty and usefulness of the present invention. This background information is not admitted prior art

The use of ethyl alcohol for purposes of motor fuel has been known since the late 1970's. However, it was not until 1998 when the United States started to increase the use of ethanol significantly. Bioethanol is a form of renewable energy that can be produced from agricultural feedstocks. It can be made from very common crops such as hemp, sugarcane, potato, cassava and corn. The majority of ethanol produced in the U.S. is made from corn, which is a cereal crop. Cereal is any grass cultivated for the edible components of its grain, composed of the endosperm, germ, and bran. Well-known cereal crops include corn (maize), rice, wheat, barley, sorghum, millet, oats and rye. Cereal grains are grown in greater quantities and provide more food energy worldwide than any other type of crop.

Referring to FIG. 1, corn ethanol is produced by means of ethanol fermentation and distillation. Ethanol is produced by microbial fermentation of the sugars in the feedstock. Microbial fermentation currently only works directly with sugars. Two major components of plants, starch and cellulose, are both made of sugars—and can, in principle, be converted to sugars for fermentation. Currently, only the sugar (e.g., sugar cane) and starch (e.g., corn) portions can be economically converted into ethanol.

There are two main types of corn ethanol production: dry milling and wet milling. Over 80% of U.S. ethanol is produced from corn by the dry grind process. Referring again to FIG. 1, the dry milling process proceeds as follows: corn grain is milled, then slurried with water to create ‘mash.’ Enzymes are added to the mash and this mixture is then cooked to hydrolyze the starch into glucose sugars. Yeast ferments these sugars into ethanol and carbon dioxide (CO2) and the ethanol is purified through a combination of distillation and molecular sieve dehydration to create fuel ethanol. The byproduct of this process is known as distiller's dried grains and solubles (DDGS) and is used wet or dry as animal feed.

There has been considerable debate about how useful bioethanol is in replacing gasoline. Concerns about its production and use relate to increased food prices due to the large amount of arable land required for crops, as well as the energy and pollution balance of the whole cycle of ethanol production, especially from corn. As such, and because most ethanol plants are in the corn belt of North America, they are subject to feedstock price volatility. In addition, the current way of doing business is to maximize the production of ethanol which is a commodity directly linked to gasoline prices, and is also subject to gasoline price volatility. Producers are required to look to different revenue streams for not only ethanol but the byproducts of the process to make ethanol such as CO2 and animal feed. In addition to ethanol and CO2 revenue streams, ethanol producers also sell dried distiller's grain from the corn.

The by-product from the fermentation process in an ethanol plant is called distiller dried grain (DDG) or distillers dried grain with solubles (DDGS) and is sold into the animal husbandry industry as a supplemental feed. The DDGS is usually devoid of starch (which is used up in ethanol production) and has a threefold increase in protein, fat, and fiber etc., compared to the original feedstock. The DDGS sold from the traditional corn ethanol plants has a protein content ranging from 22-30%, as well as other nutrients that are favorable to animal growth, such as essential amino acids, fiber and fat. However, protein content significantly higher than 30% is typically desired in animal feed.

Soymeal is the benchmark premium animal feed with a high protein content ranging from 41-48% and the required amino acids and other nutrients. Soymeal therefore commands a price substantially higher that the DDGS produced from traditional corn ethanol facilities. Soy is a legume. Legumes, which are in the family Fabaceae (or Leguminosae), are grown agriculturally primarily for their grain seed and are called pulse crops. They are grown for livestock forage and silage, and as soil-enhancing green manure. Well-known legumes include alfalfa, clover, peas, beans, chickpeas, lentils, lupin bean, mesquite, carob, soybeans, peanuts and tamarind.

Unfortunately, neither corn nor soybean crops are abundant in Canada due to Canada's cooler climate. Most soybean and soybean meal in Western Canada is shipped via rail and truck from the US, which increases the cost of such premium animal feed for Canadian livestock farmers.

Therefore, there exists a need for producing a premium animal feed with a high protein content, comparable to soybean but in a cost-effective manner in those regions of the world where soybean is not readily available.

### SUMMARY

In one embodiment, a method for producing ethanol and distiller's dried grains and solubles (DDGS) is provided. This method comprises the steps of: providing a plurality of feedstocks; milling said feedstocks; mixing said milled feedstocks into a feedstock blend; producing ethanol from said feedstock blend using a dry milling process; and collecting the DDGS byproduct, after the ethanol is produced.

In another embodiment, the method further comprises providing barley as one of the feedstocks and peas as another feedstock, wherein the barley feedstock and pea feedstock is mixed in a ratio of 2:1 to 3.5:1 to make the feedstock blend. In yet another embodiment, antifoaming enzymes are added during the dry milling process to reduce or eliminate foaming during the fermentation stage of the dry mill process.

### BRIEF DESCRIPTION OF THE DRAWINGS

FIG. 1 is a prior-art flowchart of a typical process for producing distillers dried grain with solubles (DDGS) known in the art;

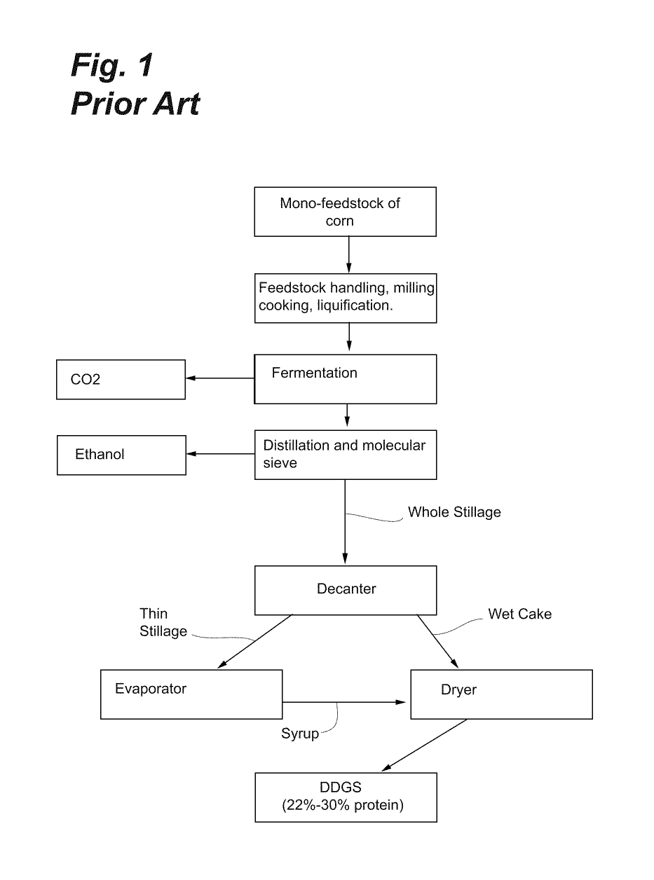

FIG. 2 is a flowchart of one embodiment of the invention for producing distillers dried grain with solubles (DDGS) having a high protein concentration; and

FIG. 3 is a flowchart of another embodiment of the invention for producing distillers dried grain with solubles (DDGS) having a high protein concentration.

### BRIEF DESCRIPTION OF THE SCHEDULES

Schedule 1 is a Technical Report—Determination of Feedstock Requirement;

Schedule 2 is a Technical Report—Nutrient Test Results;

Schedule 3 is a Technical Report—Analysis of Expected Protein Content Barley DDGS; and

Schedule 4 is a Technical Report—Pea and Barley: Evaluation of changes in the grinding and mashing procedure.

### DESCRIPTION

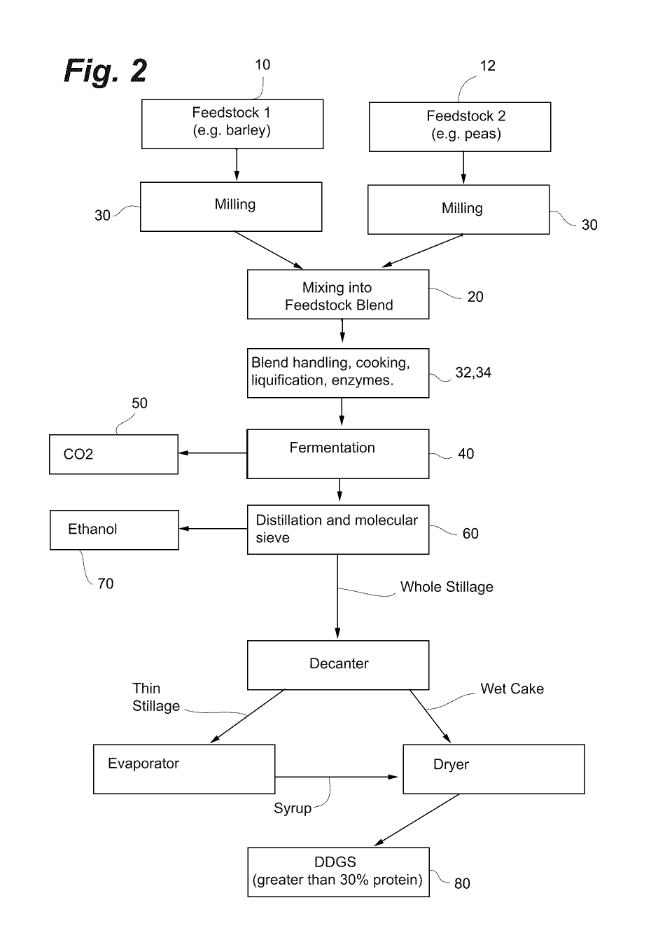

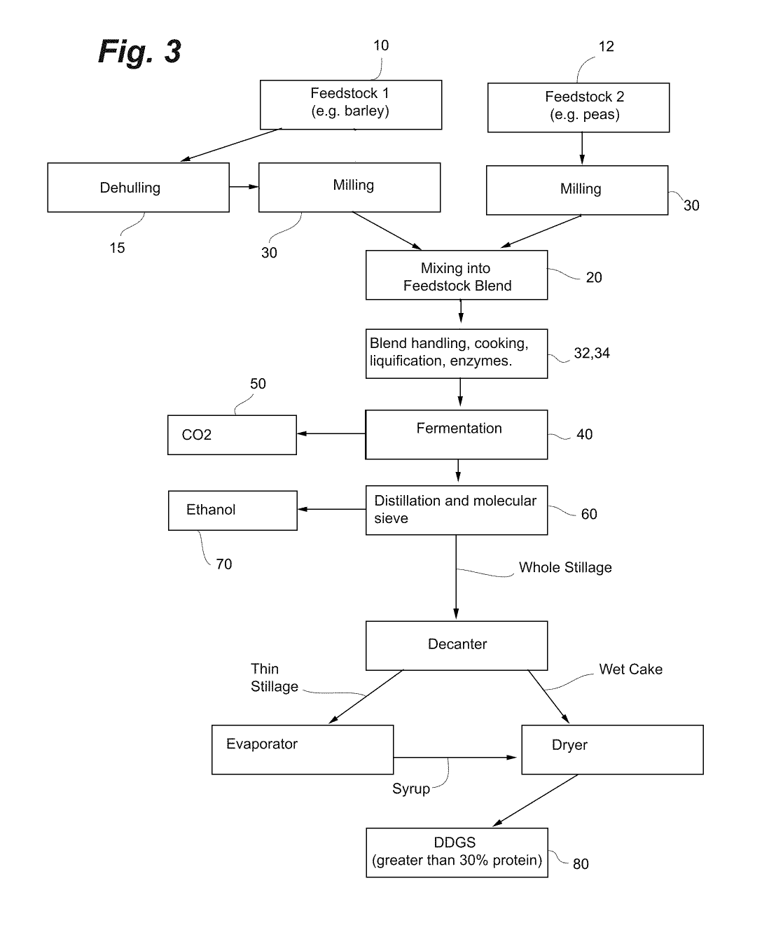

Having reference to FIGS. 2 to 3, and Schedules 1 to 4, preferred embodiments and examples for producing ethanol and a high protein distillers dried grain with solubles (DDGS) is provided.

A plurality of different grain feedstocks 10, 12 are mixed 20 into feedstock blend. The individual feedstocks are preferably each milled 30 separately prior to mixing 20 (as shown in FIGS. 2 and 3), or they may be milled 30 together as a blend after mixing 20. The milled mixture of feedstocks is then treated in the conventional “dry milling process” manner, i.e. it is slurried with water to create ‘mash’, enzymes 34 are added to the mash and this mixture is then cooked 32 to hydrolyze the starch into glucose sugars. The enzymes 34 assist with fermentation and, in certain embodiments, prevent or reduce any foaming that might otherwise occur during the fermentation stage 40. Embodiments and examples of milling, enzymes and ‘mash’ composition can be found in the Schedules (especially Schedule 4).

As is conventional, yeast ferments the sugars from the feedstock blend into ethanol and carbon dioxide (CO2) during the fermentation stage 40. The carbon dioxide (CO2) is extracted 50, and the ethanol may be further purified through a combination of distillation and molecular sieve dehydration 60 to create fuel ethanol 70. The distiller's dried grains and solubles (DDGS) 80 that is then produced at the end stage will have a higher protein content than is the case when only a mono crop feedstock, such as corn, is used.

Advantageously, feedstocks such as wheat, barley, peas, and canola are much more prevalent in Canada than are soy and corn. More advantageously, barley is currently known as an inexpensive, low value feed crop. Utilizing the inventor's various embodiments with barley as at least one of the feedstocks 10 to make the feedstock blend 20, results in cost savings, when producing ethanol and DDGS. More advantageously, by not using the conventional corn as a feedstock, price volatility of the corn market is avoided. Even more advantageously, the higher protein concentration in the distiller's dried grains and solubles (DDGS) 80 maximizes the monetary value of this by-product from the ethanol production process.

The inventors have found that blending barley as the first feedstock 10, with peas as the second feedstock 12, at a ratio of between 2:1 to 3.5:1 by weight in the ethanol production process provided herein 20, 30, 32, 34, 40, 50, 60, results in conventional quantities of ethanol production 70, and also in a distiller's dried grains and solubles (DDGS) 80 with a high protein concentration, typically of at least 40%. As shown in the Schedules, the protein profile of the DDGS 80 (resulting from a 3:1 (barley:peas) feedstock blend using the one or more embodiments of the invention results) produced is very similar to the protein profile of DDGS that would otherwise be produced using much more expensive soybeans.

By using such a feedstock blend 10, 12, the invention maximizes total revenues and on a comparable basis to a traditional corn-only based ethanol plant; and the invention may yield revenues 50% higher than doing business the conventional corn-only way. In addition, the barley and pea feedstock price volatility is much lower than corn feedstock price volatility.

The inventors have also discovered that by using 2:1 to 3:1 blends of corn 10 with peas 12 as the ethanol feedstocks, that the protein and nutrient value of the by-product DDGS 80 is similarly improved. While such a corn/pea blend based ethanol production may still be subject to the volatility of the corn feedstock market, the end product DDGS 80 will advantageously have a higher quality protein profile, similar to a DDGS produced based on soybeans as a feedstock.

Referring to FIG. 3, another embodiment for producing ethanol and a high protein distillers dried grain with solubles (DDGS) is provided. In this embodiment, barley 10 and peas 12 are again provided as the feedstocks to make the feedstock blend 20. However, prior to milling and blending with the peas 12, the barley 10 is dehulled (also called debranning or decortication) during a dehulling step 15 using conventional dehulling equipment and techniques. This dehulling 15 removes the barley hulls which are a known carrier of Fusarium fungus. Fusarium is a common mold on most grain crops and is toxic to swine. As such, Fusarium contamination is a major issue for cereal-based ethanol plants—because the fusarium is not killed during fermentation and further processing. By removing the barley hulls prior to mixing 20 and milling 30, Fusarium contamination is avoided, and the resultant DDGS 80 will not only have a higher protein concentration than conventional DDGS, but will also be safe as a feed for swine.

In another embodiment (not shown in the Figures, but described in the Schedules), the pea feedstock is dehulled, prior to milling.

### EXAMPLES

Having reference to Schedule. 2, which shows results from the inventor's research and testing at lab scale, and then in a commercial scale test in an operating ethanol facility in the USA. The commercial test confirmed the enzymes to be used in the production process, ethanol yields and the nutrient content (protein levels averaging 42-44% and a very good amino acid profile) of the DDGS, which was branded “Prairie Gold” (see Schedule. 2). The PRAIRIE GOLD™ DDGS was then used in animal feed trials at the Hog and Swine Institute at the University of Saskatchewan, further confirming this DDGS nutrient value and animal health benefits. This combination of ethanol production and DDGS yields substantially higher project revenues compared to today's existing ethanol production facilities.

The specific combination of barley and peas provided herein (see Schedules), increases the protein content of the DDGS 80 to approximately 44%, making it a high value product for the swine, dairy, poultry and aquaculture industries. The protein level of the PRAIRIE GOLD™ DDGS 80 will allow it to compete directly against U.S. soybean meal as an animal feed. Most soybean and soybean meal in Western Canada is shipped via rail and truck from the U.S., thereby making a locally grown DDGS 80 feed source even more economical and valuable.

In summary, various embodiments of the invention include the following components:
a) Identification that by blending peas with traditional feedstock, such as corn and wheat, in a traditional ethanol plant using fermentation will significantly increase the protein content and value of the DDG and DDGS by-product;
b) That an optimal blend of dehulled barley 10 and field peas 12 will maximize the value of the DDG and DDGS produced in fermentable ethanol production facilities;
c) Identification that de-hulling barley removes Fusarium in the grain; and
d) Identification of the appropriate commercially available enzyme to maximize production from barley and pea feedstock blend.

Additional examples are described in the Schedules.

Those of ordinary skill in the art will appreciate that various modifications to the invention as described herein will be possible without falling outside the scope of the invention. In the claims, the word “comprising” is used in its inclusive sense and does not exclude other elements being present. The indefinite article “a” before a claim feature does not exclude more than one of the features being present.

1. A method for producing ethanol and distiller's dried grains and solubles (DDGS) comprising:
providing a plurality of feedstocks;
milling said feedstocks;
mixing said milled feedstocks into a feedstock blend;
producing ethanol from said feedstock blend using a dry milling process;
collecting the DDGS byproduct, after the ethanol is produced.

2. The method according to claim 1 wherein the plurality of feedstocks comprises at least one feedstock comprised of peas.

3. The method according to claim 2 wherein the plurality of feedstocks comprises at least one feedstock comprised of barley.

4. The method according to claim 1 wherein the plurality of feedstocks comprises at least a first feedstock of peas and a second feedstock of barley.

5. The method according to claim 4, wherein the feedstock of barley is dehulled prior to milling.

6. The method according to claim 4, wherein the feedstock of peas is dehulled prior to milling.

7. The method according to claim 4, wherein both the feedstock of barley and peas are dehulled prior to milling.

8. The method according to claim 4, wherein the feedstocks of barley and peas are provided in a ratio between 2:1 to 3.5:1 by weight.

9. The method according to claim 8, wherein the feedstock of barley is dehulled prior to milling.

10. The method according to claim 8, wherein the feedstock of peas is dehulled prior to milling.

11. The method according to claim 8, wherein both the feedstock of barley and peas are dehulled prior to milling.

12. The method according to claim 8, wherein antifoaming enzymes are added to the feedstock blend, to reduce or eliminate foaming during the dry milling process.
